(19) United States
(12) Patent Application Publication
(10) Pub. No.: US 2019/0328420 A1
(43) Pub. Date: Oct. 31, 2019

(54) PERICARDIAL SPACE ACCESS DEVICE AND METHOD

(71) Applicants: Sebastian Khairkhahan, Palo Alto, CA (US); Alexander Khairkhahan, Palo Alto, CA (US)

(72) Inventors: Sebastian Khairkhahan, Palo Alto, CA (US); Alexander Khairkhahan, Palo Alto, CA (US)

(21) Appl. No.: 16/368,644

(22) Filed: Mar. 28, 2019

Related U.S. Application Data
(60) Provisional application No. 62/664,785, filed on Apr. 30, 2018.

(51) Int. Cl. A61B 17/34

(52) U.S. Cl. CPC A61B 17/3423; A61B 2017/00991; A61B 2017/3488; A61B 2017/3425

(57) ABSTRACT

A pericardial access device includes an outer tube, an inner tube, a pericardial interface structure, and an access needle. The inner tube is axially, slidably received in an axial passage of the outer tube. The pericardial interface structure is disposed on a distal end of the inner tube and has a plurality of tissue-engaging tines which deploy from a constrained configuration to a deployed configuration as the inner tube is advanced distally through the outer tube. The plurality of tissue-engaging tines has been adapted to engage the parietal pericardium to draw the parietal pericardium away from the visceral pericardium to enlarge a pericardial space to improve access. Alternatively the plurality of tines can be adapted to atraumatically engage the parietal pericardium to reduce the risk of advancing a cannula having a tissue-penetrating distal end. The access needle has a tissue-penetrating tip on a distal end thereof and is slidably received in an axial passage of the inner tube.

### CROSS-REFERENCE TO RELATED APPLICATION

This application claims the benefit of provisional application 62/664,785 (Attorney Docket No. 54317-703.101), filed on Apr. 30, 2018, the full disclosure of which is incorporated herein by reference.

### BACKGROUND OF THE INVENTION

### 1. Field of the Invention

The present invention relates generally to medical devices and methods. More specifically, the present invention relates to devices and methods establishing access to a pericardial space.

The human heart is enveloped within a tissue structure referred to as the pericardium. The pericardium includes two major layers. The layer which lies immediately over the epicardial surface of the heart is referred to as the visceral pericardium. The second layer is formed as a sac around the visceral pericardium and is referred to as the parietal pericardium. Normally, the visceral and parietal pericardia lie in close contact with each other and are separated only by a thin layer of pericardial fluid. The space between the visceral and parietal pericardia is a “potential” space referred to as the pericardial space.

Access to the pericardial space is useful for a variety of purposes including the placement of epicardial pacing leads, drug delivery, transmyocardial access to the heart chambers, and of particular interest to the present invention, cardiac ablation. Surgical access can be obtained via an open sternotomy where the patient's sternum is divided and the parietal pericardium exposed. Such an approach, however, is highly traumatic, requiring general anesthesia and useful only under compelling circumstances.

Access to the pericardial space can also be achieved less invasively using a thoracoscopic approach where access is established via an introducer sheath positioned through the parietal pericardium and into the pericardial space over the visceral pericardium. .

It would be desirable to provide additional and improved methods and apparatus for the minimally invasive access to a patient's pericardial space. The methods and devices should be suitable for a wide variety of minimally invasive approaches to the pericardium, including at least intercostal/transthoracic and subxiphoid approaches, and the like. The methods and devices should further provide for secure and stable capture of the parietal pericardium and permit the opening of a large space or volume between the parietal and visceral pericardia. At least some of these objectives will be met by the inventions described and claimed below.

### 2. Listing of the Background Art

Relevant US Patents and Patent Publications include: US2008/294174; U.S. Pat. Nos. 9,259,317; 9,179,932; 8,986,278; 8,603,031; 8,460,181; 8,317,810; 7,881,810; 7,736,347; 6,835,193; 6,156,009; US2017/119435; US2015/230699; and US2005/234507.

### SUMMARY OF THE INVENTION

The present invention provides devices, systems, and methods for initiating and establishing pericardial access for performing intra-pericardial procedures, trans-myocardial procedures including fluid withdrawal, drug delivery, diagnostic and therapeutic electrophysiology procedures, pacemaker lead implantation, defibrillator lead placement, placement of the left ventricular assist devices, placement of the arterial bypass grafts, in situ bypass, i.e., coronary artery-venous fistulae, placement of drug delivery depots, closure of the left arterial appendage, and the and the like. The present invention is particularly useful for cardiac ablation procedures when ablation catheters can be advanced over guidewires placed into the pericardial space by the methods and devices of the present invention.

In a first aspect, the present invention provides a pericardial access device which includes an outer tube, an inner tube, and an access needle. The outer tube has an axial passage which is configured to slidably receive the inner tube to allow a distal end of the inner tube to be axially extended and retracted relative to a distal end of the outer tube. A pericardial interface structure is formed or disposed on a distal end of the inner tube and typically includes a plurality of tissue-engaging tines, where the tines are configured to deploy from a constrained configuration to an expanded configuration as the pericardial interface structure is advanced distally from the distal end of the outer tube.

Typically, individual tissue-engaging tines are formed from an elastic or superelastic material and are in their “deployed” configuration, typically curved over an arc of 150° to 210°, when unconstrained. Prior to deployment, however, the tines are usually constrained within a distal region of the outer tube in a low profile configuration which facilitates introduction and removal of the pericardial access device from a target location, often including advancement through a laparoscopic, thoracoscopic, or similar minimally invasive introducing port. The access needle typically includes a tissue-penetrating tip on its distal end, where the access needle is slidably received in an axial passage of the inner tube. In this way, after the pericardial access device is introduced to a surgical target site, the inner tube can be advanced distally from or retracted proximally back into the outer tube, depending on the particular treatment protocol. The access needle can be advanced from the inner tube to penetrate a pericardial or other tissue site after the tissue-engaging tines are deployed in the desired manner.

In specific examples, the individual tissue-engaging tines are further configured to penetrate tissue as they are advanced from the distal end of the outer tube. In such instances, as described more fully below, tines may be used to engage and retract a parietal pericardium of the patient's heart in order to enlarge a pericardial space to perform procedures within the pericardial space. In alternative instances, however, the plurality of tissue-engaging tines may be configured to atraumatically engaged tissue as they are advanced from a distal end of the outer tube. In such instances, the devices of the present invention will typically not be used to retract the parietal pericardium in order to enlarge a pericardial space prior to needle deployment into the pericardial space. In still other instances, the tissue-engaging tines may be configured to permit both tissue-penetrating engagement with the parietal pericardium and atraumatic engagement with the parietal pericardium so that the same access device can be used in different pericardial space access protocols.

In specific embodiments of the devices of the present invention, the plurality of tissue-engaging tines will be disposed about a periphery of a distal end of the inner tube. In such instances, each tine will typically be straightened when constrained within the outer tube and will evert radially outwardly as the tine is advanced from the distal end of the outer tube. In some instances, the tines will have tissue-penetrating tips so that they will be engaged against the parietal pericardium to penetrate and capture the parietal pericardium in order to retract it to enlarge the pericardial space. In other instances, the leading or distal surfaces of the everted tines may provide an atraumatic tip for engaging the parietal pericardium without tissue penetration. In some instances, whether or not the tines penetrate the parietal pericardium will depend on whether or not the tips of the tines are engaged against the parietal pericardium while they are being advanced from the other tube.

In preferred embodiments of the present invention, the outer tubes may comprise a cannula having a blunt end such that the cannula can be engaged directly against the parietal pericardium or other tissue surface as the inner tube is being advanced to deploy the tissue-engaging tines. The use of cannulas having a blunt tip to engage the parietal pericardium is particularly advantageous when the tines are being used to penetrate and capture the parietal pericardium in order to enlarge the pericardial space.

In other instances, the outer tube may possess a tissue-penetrating distal end. Such embodiments are useful for allowing the access system to self-penetrate the patient's skin in order to provide direct percutaneous access, e.g. sub-xiphoid or intercostal access to the patient's heart. In such instances, the outer tube will typically be retracted over the inner tube and atraumatic-tissue engaging tines or other structures will be deployed from the inner tube prior to engaging a pericardial surface and percutaneously advancing the tissue-penetrating tip of the outer tube through target tissue.

In still other embodiments and instances, the inner tube may have an adjustable needle-stop structure at its proximal end in order to control a penetration distance afforded the access needle tip. For example, a threaded end cap may be placed at a proximal end of the inner tube in order to provide an axially adjustable needle stop.

In a second aspect, the present invention provides a method for accessing and enlarging a pericardial space between a visceral pericardium and a parietal pericardium surrounding a patient's heart. The method comprises advancing an atraumatic end of a cannula against a target site on an exterior of the parietal pericardium. An inner tube is advanced through an axial passageway on the cannula to advance a plurality of tines from a distal end of the cannula, where individual tines evert and penetrate the parietal pericardium, resulting in capture of the parietal pericardium by the inner tube. After the parietal pericardium has been captured, the cannula and inner tube may be drawn proximally, i.e. in a direction away from the surface of the heart, to separate the parietal pericardium from the underlying visceral pericardium, resulting in enlargement of the pericardial space. After the pericardial space has been enlarged, an access needle may be advanced through an axial passage of the inner tube so that a distal tip of the access needle enters the enlarged pericardial space with minimal or no penetration of the visceral pericardium or underlying myocardium. Once in place, the needle has a lumen to provide an access path into the enlarged pericardial space to allow a variety of procedures, such as drug delivery, cardiac ablations, and other procedures as noted in the background above.

In particular aspects of the methods herein, a guidewire will typically be advanced through a lumen of the access needle to a target location in the parietal space. After the guidewire is in place, the pericardial access system, including thecannula, inner tube, and access needle, may be retracted or withdrawn over a proximal section of the guidewire, allowing an introducer sheet to be advanced over the guidewire and through the parietal pericardium to establish working access into the pericardial space.

In a third aspect, the present invention provides a second method for accessing a pericardial space between a visceral pericardium and a parietal pericardium. This second method comprises percutaneously advancing a tissue-penetrating end of a cannula (outer tube) into a patient's thorax. A tissue-penetrating distal tip of the cannula is then retracted proximally over an inner tube to cause or allow a plurality of tines to evert radially outwardly from a distal end of the inner tube to form an atraumatic interface over a target site on an exterior of the parietal pericardium. The inner tube is then advanced through the retracted cannula to engage the atraumatic interface against the target site on the exterior of the parietal pericardium. An access needle may then be advanced through an axial passage of the inner tube so that a distal tip of the needle passes through the parietal pericardium and enters the pericardial space with limited or no penetration of the visceral pericardium. The needle has an access lumen, preferably on a side of a distal tip of the needle immediately proximal to a tissue-penetration tip of the needle, to provide an access path into the pericardial space.

The needle may then be used to introduce a guidewire, introduce drugs, or for any other purpose conventionally performed within the pericardial space.

### BRIEF DESCRIPTION OF THE DRAWINGS

FIG. 1 is an exploded view of a pericardial access system constructed in accordance with the principles of the present invention.

FIGS. 2A through 2D illustrate assembly and use of the components of the pericardial access system of FIG. 1.

FIGS. 3A through 3E illustrate use of the pericardial access system of FIG. 1 for penetrating and enlarging a pericardial space in the heart of a patient in accordance with the principles of the methods of the present invention.

FIGS. 4A through 4E illustrate the advancement of a guidewire into a pericardial space of a patient enlarged by the methods and tools described herein for use in a variety of cardiac procedures.

FIGS. 5A through 5D illustrate use of a guidewire which has been introduced by the methods of the present invention for advancing a secondary guidewire around the pulmonary veins of a patient for use in cardiac ablation or other procedures.

FIG. 6 illustrates an alternative embodiment of a pericardial access system constructed in accordance with the principles of the present invention having a tissue-penetrating outer tube and an atraumatic pericardial interface structure.

FIGS. 7A through 7D illustrate use of the pericardial access system of FIG. 6 for penetrating a pericardial space without enlargement and advancing a guidewire into that pericardial space.

### DETAILED DESCRIPTION OF THE INVENTION

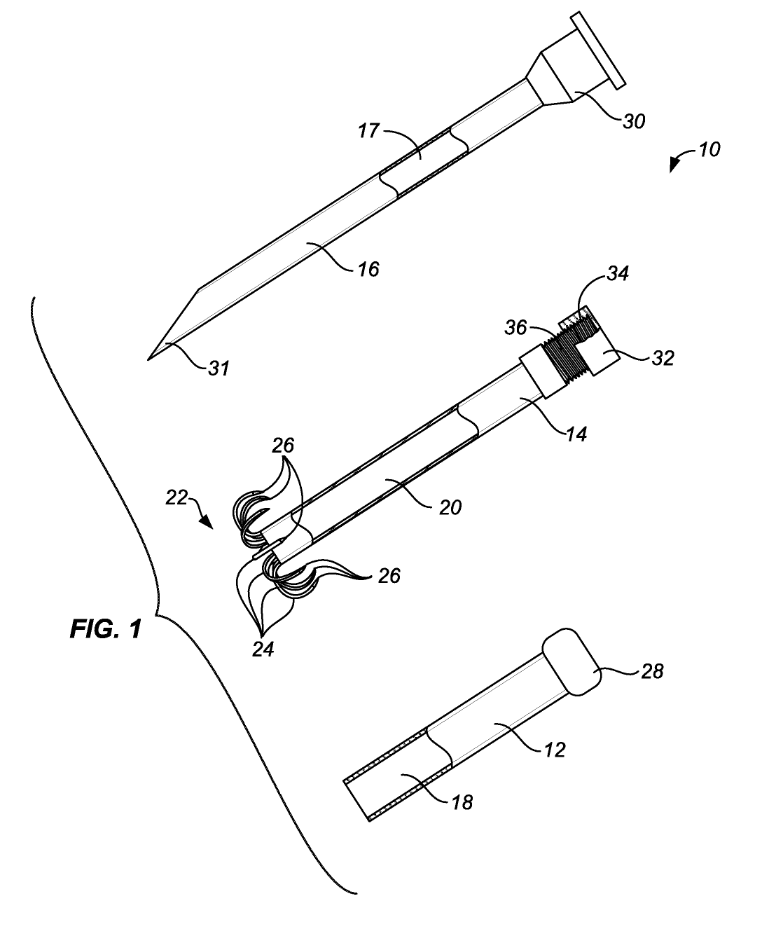

Referring to FIG. 1, a pericardial access system 10 constructed in accordance with the principles of the present invention comprises an outer tube 12, an inner tube 14, and an access needle 16. The outer tube 12 has an axial passage 18 extending therethrough. Similarly, the inner tube 14 has an axial passage 20 extending therethrough. The access needle 16 is hollow and will also include an inner lumen 17.

The inner tube 14 further includes a pericardial interface structure 22 formed on or otherwise coupled to a distal end of the tube. Pericardial interface structure 22 typically comprises a plurality of tissue-engaging tines 24, where the tines may have tissue-penetrating tips 26, at least in the first embodiment illustrated in FIG. 1. The tissue-engaging tines 24 are typically elastic or resilient, often being formed from an elastic or superelastic metal. In specific embodiments, individual tines 24 will be curved when unconstrained so that the tines may evert after release from constraint, as described hereinbelow.

The outer tube 12 typically comprises a hub 28 or other feature allowing manual manipulation in the outer tube. The access needle 16 will also include a hub 30 at its proximal end. The distal end or tip of the needle 16 will be sharpened or otherwise formed into a tissue-penetrating tip 31.

A proximal end of the inner tube 14 will preferably be formed with an adjustable needle stop 32. By adjustable, it is meant that the needle stop will be axially positionable relative to the remainder of the inner tube 20. By selecting an axial position of the needle stop 32, the advancement of the needle can be controlled as the needle stop will block further advancement of the hub 30 of the access needle 16. Usually, the axial adjustability of the needle stop 32 is achieved with a threaded connection, e.g. with internal threads 34 on the needle stop 32 (which is formed as a cylindrical cap) which engage external threads 36 on a proximal end of a main body of the inner tube 20.

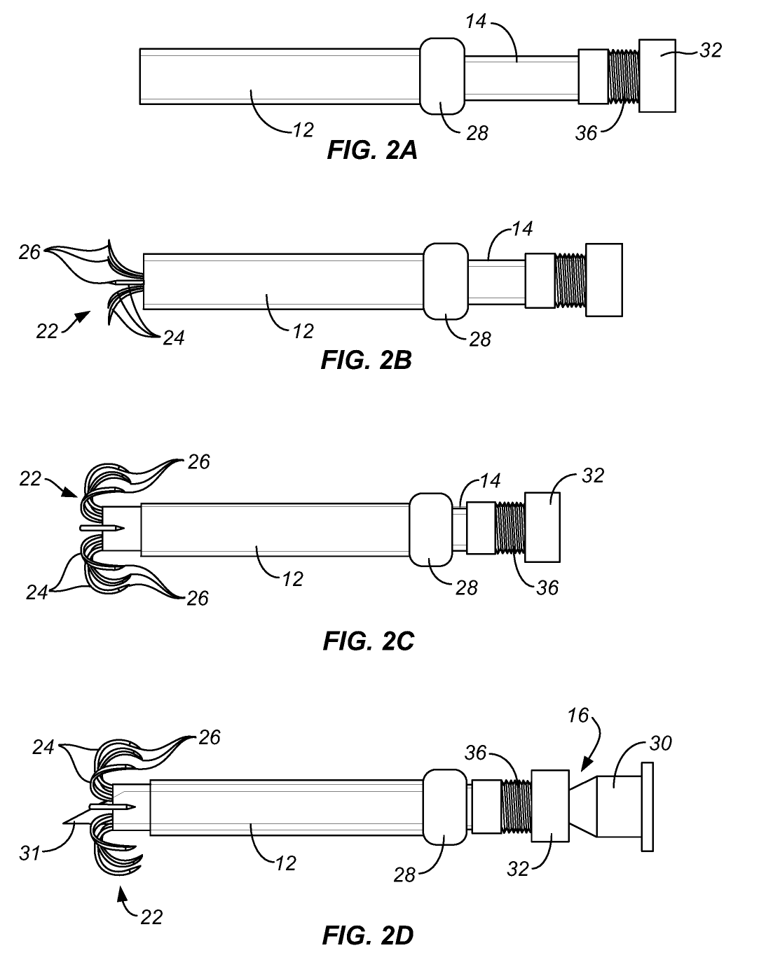

As shown in FIGS. 2A through 2D, the inner tube 14 is typically received in the axial passage 18 (FIG. 1) of the outer tube 12. In an initial state or condition, the pericardial interface surface 22 will be restrained within a distal end of the outer tube 12, as shown in FIG. 2A.

By axially advancing the inner tube 14 relative to the outer tube 12, the tissue-engaging tines 24 of the pericardial interface structure 22 will emerge from the distal end of the outer tube. As the tines 24 emerge, they will evert in a radially outward direction, with sharpened tips 26 of the tines leading of the way. In this way, as described in more detail later, the sharpened tips may penetrate and capture the parietal pericardium or other tissue surface as the tines are advanced.

As the inner tube 14 is further axially advanced in a distal direction, as shown in FIG. 2C, the distal end of the inner tube 12 will fully emerge, allowing the tines 24 of the pericardial interface structure 22 to fully deploy and evert.

As shown in FIG. 2D, the needle 16 can be advanced through the inner tube 14 of the pericardial access system 10 so that the distal tip 31 emerges from the distal end of the inner tube beyond a distal-most region or surface of the pericardial interface structure 22. The extent (distance) to which the sharp tip 31 emerges from the inner tube 14 depends on the position of the needle stop 32 which can be adjusted by rotating the needle stop on the external threads 36 of the inner tube.

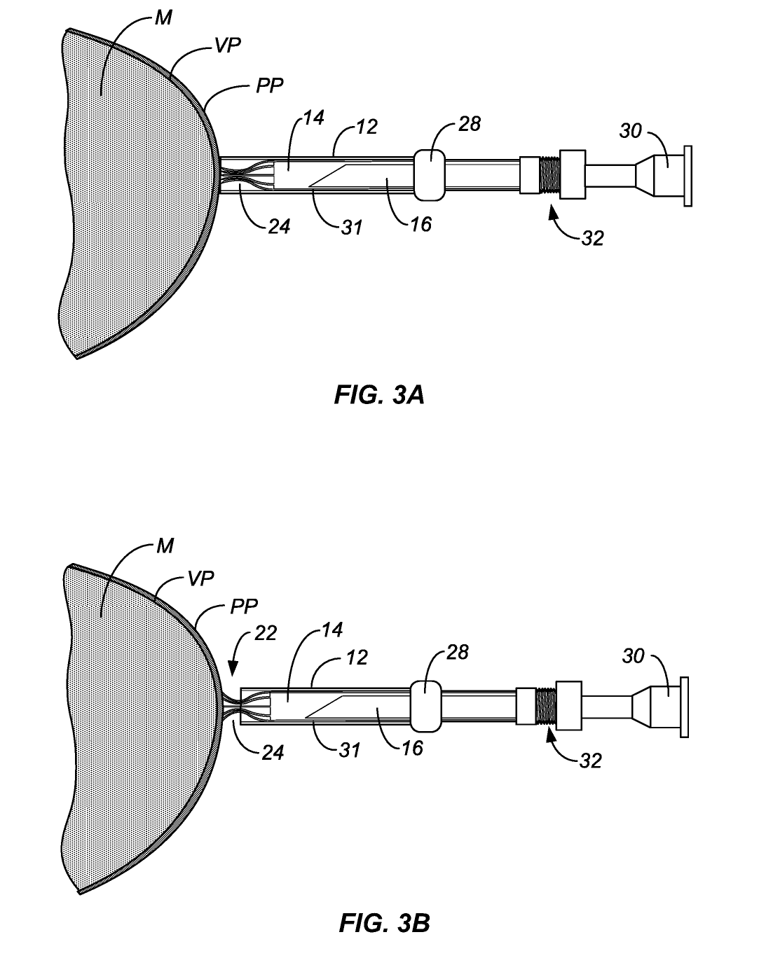

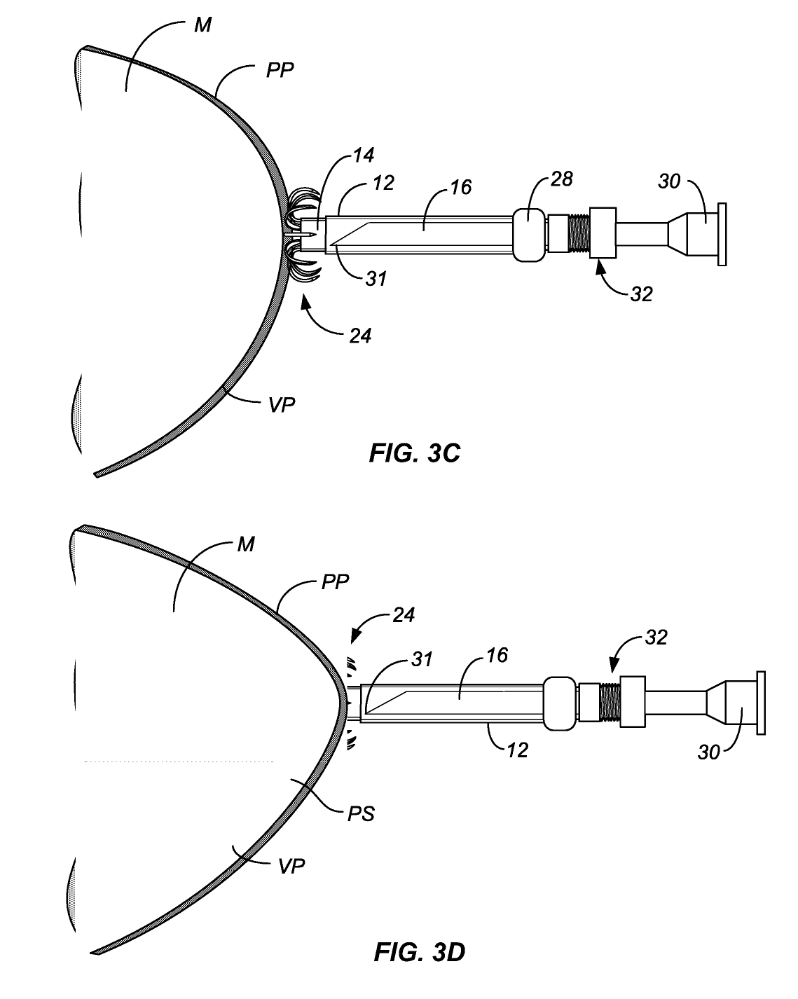

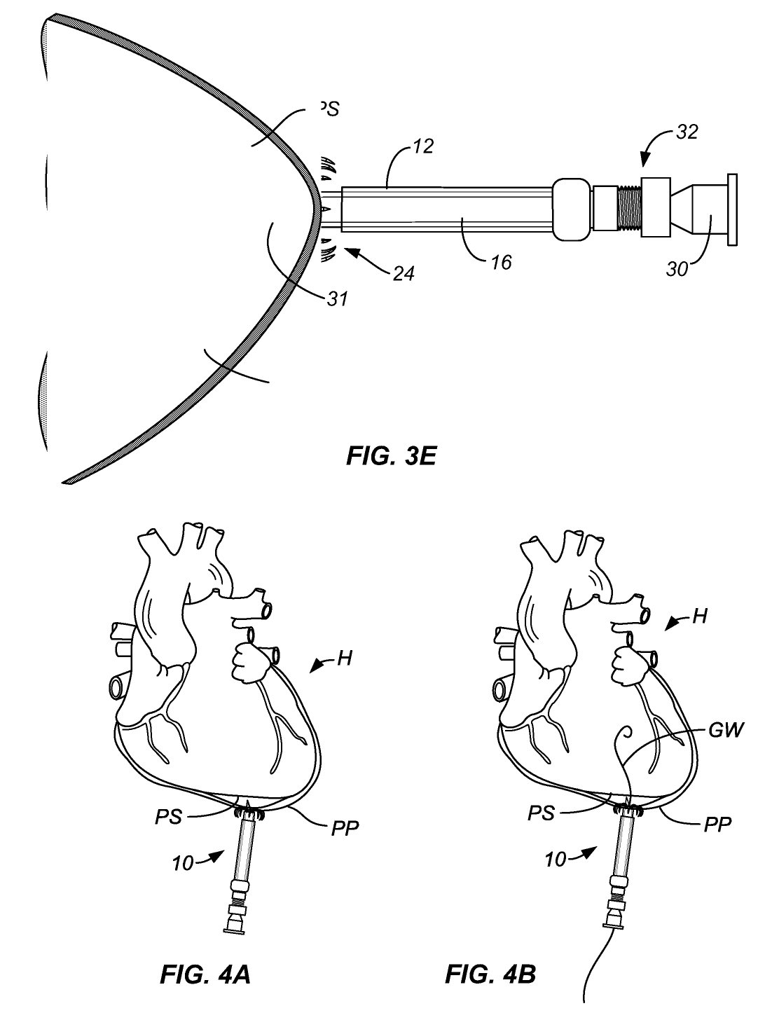

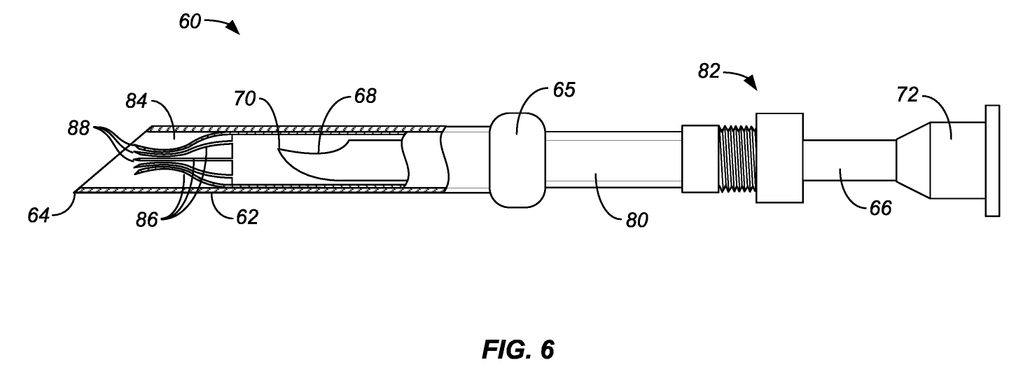

Referring now to FIGS. 3A through 3E, use of the pericardial access system 10 for accessing an enlarged pericardial space will be described. As shown in FIG. 3A, a distal end of the outer tube 12 is initially engaged against a target location on the exterior of the parietal pericardium PP. For such access, it is desirable that the distal end of the outer tube be atraumatic, e.g. iusing a cannula having an atraumatic distal tip. In particular, it is desirable not to use an outer tube with a tissue-penetrating tip as shown, for example, in the embodiments of FIG. 6 described below.

After the outer tube 12 initial contacts the parietal pericardium, the inner tube 14 is axially advanced so that the tissue engaging tines 24 of the pericardial interface structure 22 emerge and begin to evert, as show in in FIG. 3B. As the distal tips 26 of the tissue-engaging tines 24 are typically sharpened into tissue-penetrating points (FIG. 1), the tips will penetrate the parietal pericardium PP with minimal or no penetration into the underlying visceral pericardium VP, as shown in FIG. 3C.

Once the tissue-penetrating tines 24 have fully everted, as shown in FIG. 3C, the entire assembly of the pericardial access system 10 can be retracted so that the parietal pericardium PP can be drawn away from the visceral pericardium VP to create an enlarged pericardial space PS, as shown in FIG. 3D.

Once the enlarged pericardial space PS has been created, the sharpened tip 31 of the needle 16 can be advanced to penetrate the parietal pericardium PP, as shown in FIG. 3E. The lumen in the needle is then available for introducing tools, drugs, guidewires, or for any other purpose facilitated or enabled by needle access into an enlarged pericardial space.

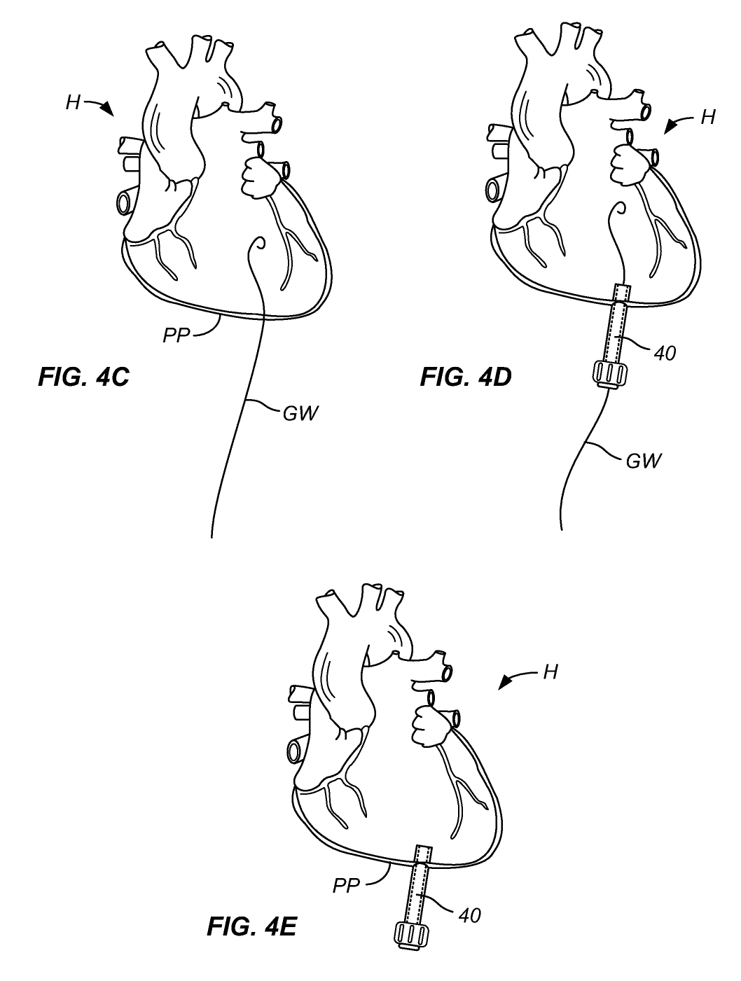

For example, as shown in FIGS. 4A through 4E, a guidewire GW may be introduced into the pericardial space PS and advanced to a desired location. Usually, as shown in FIG. 4A, pericardial access system is used to capture and pull the parietal pericardium PP away from the visceral pericardium VP to provide the enlarged pericardial space PS, as described. A guidewire GW can then be introduced through the pericardial access system 10 and advanced into the pericardial space PS, as shown in FIG. 4B. Once the guidewire GW is sufficiently advanced into the pericardial space PS, the pericardial access system may be proximally withdrawn over the proximal section of the guidewire GW, as shown in FIG. 4C, leaving the guidewire GW in place for subsequent use. As shown in FIG. 4D, a conventional access tube 40 may then be introduced over the guidewire GW and advanced into the pericardial space PS to provide access for other tools or purposes, as shown in FIGS. 4D and 4E.

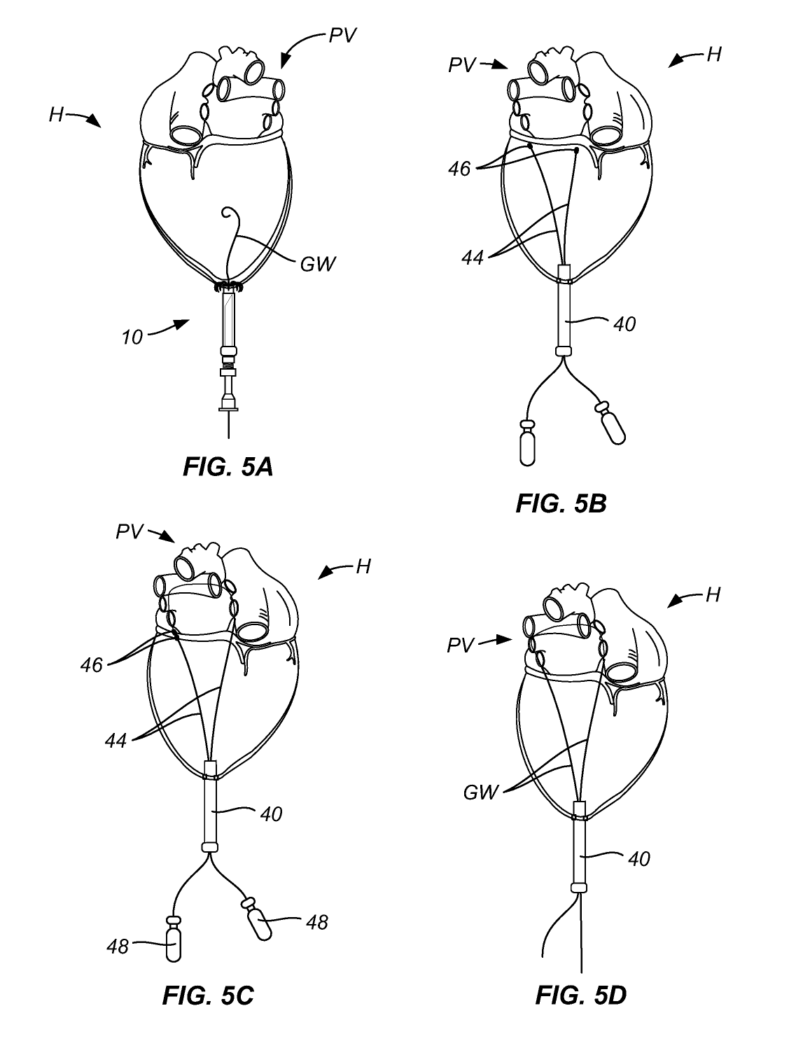

Referring now to FIGS. 5A though 5D, the guidewire GW introduced into through the pericardial access system 10 (as shown in FIG. 5A), may be used to introduce an access sheath 40 which in turn may be used to introduce a pair of deflectable guidewires GW into the pericardial space PS and toward the pulmonary veins PV, as shown in FIG. 5B. Using control handles 48 for steering, one or both of the guidewires 44 may be looped around the pulmonary veins PV and the magnets 46 coupled together, as shown in FIGS. 5C. The magnetic guidewires 44 may be then exchanged for a continuous guidewire GW, as shown in FIG. 5D, and the continuous guidewire used for a variety of purposes, such as for advancing an ablation catheter to perform pulmonary vein ablation.

Referring to FIG. 6, an alternative embodiment of a pericardial access system 60 constructed in accordance with the principles of the present invention will be described. The pericardial access system 60 comprises an outer tube 62. The outer tube 62 differs from outer tube 12 of pericardial access system 10 in that outer tube 62 has a tissue-penetrating tip 64 which allows self-introduction of the pericardial access system 60.

The pericardial access system 60 further includes a hub 65 at the proximal end of the outer tube 62 and an access needle 66 having a side port 68 adjacent to a tissue-penetrating distal tip 70. Inner tube 80 of pericardial access system 60 may be similar to inner tube 12 of pericardial access system 10. Typically, inner tube 80 will include a pericardial interface structure 84 at its distal end, where the interface structure includes a plurality of tissue-engaging tines 86 having distal tips 88. While in some instances the distal tips 86 might be sharpened or otherwise self-penetrating, the tissue interface structure 84 is intended primarily to be atraumatic, so the distal tips will often be blunt or otherwise made atraumatic to limit penetration into or other injury of the pericardium.

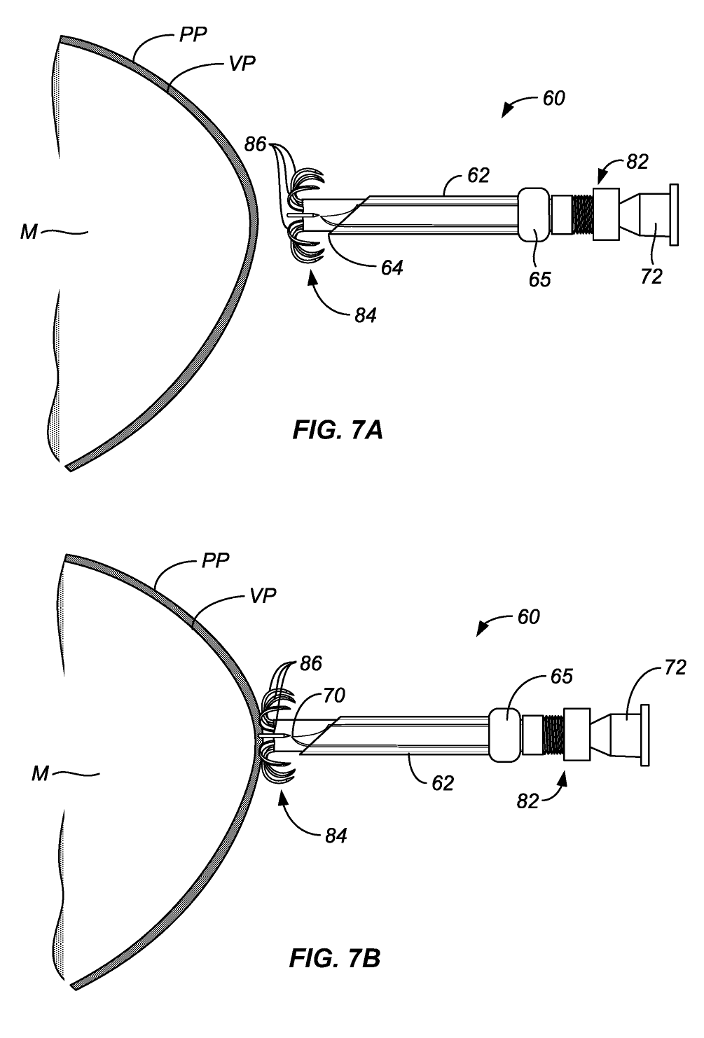

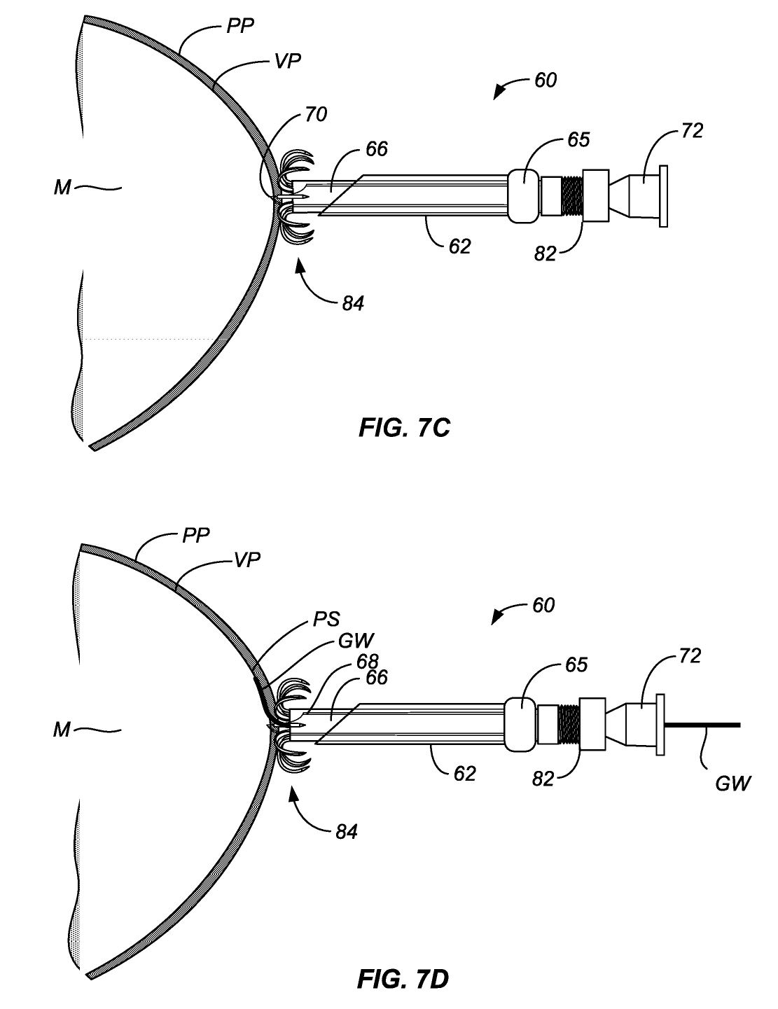

Pericardial access system 60 may be used to access a pericardial space as shown in FIG. 7A through 7D. Initially, the assembly of the pericardial access system 60, as shown for example in FIG. 6, may be self introduced percutaneously through a target site in the patient's thorax, for example, in a sub-xiphoid approach or an intercostal approach. Once the assembly of the pericardial access system has been introduced, the sharpened distal end 64 of the outer tube 60 will be retracted over the inner tube 80, as shown in FIG. 7A. Such retraction will simultaneously cause deployment of the tissue-engaging tine 86 of the pericardial interface structure 84. In contrast to previously described protocols, however, the deployment of the pericardial interface structure 84 will be done above the pericardium and the heart so that the distal tips 88 of the tines 86 do not penetrate the pericardium.

Once the pericardial interface structure 84 is deployed, a front or leading segment of the interface structure may be atraumatically engaged against the exterior of the pericardium, as shown in FIG. 7B. The access needle 66 may then be advanced so that the tissue-penetrating tip 70 penetrates the parietal pericardium PP, as shown in FIG. 7C. Usually, minimal or no penetration of the underlying visceral pericardium VP or myocardium M will be effected. The side port 68 on the access needle 66 allows a guidewire GW to be laterally deployed into the pericardial space PS even without enlargement of the space. The guidewire can then be advanced to a desired location and then the pericardial access system 60 then exchanged for a conventional access tube as described previously.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A pericardial access device comprising:
an outer tube;
an inner tube axially slidably received in an axial passage of the outer tube;
a pericardial interface structure disposed on a distal end of the inner tube and having a plurality of tissue-engaging tines configured to deploy from a constrained configuration to a deployed configuration as the inner tube is advanced distally through the outer tube; and
an access needle having a tissue-penetrating tip on a distal end thereof, said needle being slidably received in an axial passage of the inner tube.

2. The pericardial access device of claim 1 wherein the plurality of tissue-engaging tines are further configured to penetrate tissue as they are advanced from a distal end of the outer tube.

3. The pericardial access device of claim 1 wherein the plurality of tissue-engaging tines have tissue-penetrating distal tips.

4. The pericardial access device of claim 1 wherein the plurality of tissue-engaging tines are further configured to atraumatically engage tissue as they are advanced from a distal end of the outer tube.

5. The pericardial access device of claim 1 wherein the plurality of tissue-engaging tines are disposed about a periphery of a distal end of the inner tube and wherein each tine is straightened when constrained in the outer tube and everts radially outwardly as it is advanced from the distal end of the outer tube.

6. The pericardial access device of claim 1 wherein the outer tube comprises a cannula having a blunt end.

7. The pericardial access device of claim 1 wherein the outer tube comprises a needle having a tissue-penetrating end.

8. The pericardial access device of claim 1 further comprising an adjustable needle stop mechanism to adjustably limit advancement of the access needle from a distal end of the inner tube.

9. The pericardial access device of claim 8 wherein the needle stop mechanism comprises a threaded end cap.

10. The pericardial access device of claim 1 wherein the inner needle has a side exit hole.

11. The pericardial access device of claim 1 further comprising a guidewire configured to be advanced through the access needle into a pericardial space.

12. A method for accessing a pericardial space between a visceral pericardium and a parietal pericardium surrounding a patient's heart, said method comprising:
advancing an atraumatic end of a cannula against a target site on an exterior of the parietal pericardium surrounding the patient's heart;
advancing an inner tube through an axial passageway of the cannula to cause a plurality of tines to exit a distal end of the cannula and to evert and penetrate the parietal pericardium;
drawing proximally on the cannula and inner tube to separate the parietal pericardium from the visceral pericardium and enlarge the pericardial space;
advancing an access needle through an axial passage of the inner tube so that a distal tip of the access needle enters the enlarge pericardial space with minimal or no penetration of the visceral pericardium, wherein the needle has a lumen to provide an access path into the enlarged pericardial space.

13. The method of claim 12 further comprising advancing a guidewire through a lumen of the access needle to a target location in the peritoneal space.

14. The method of claim 13 further comprising withdrawing the cannula, inner tube, and access needle from over a proximal section of the guidewire and advancing an introducer sheath over the proximal section of the guidewire and through the parietal pericardium to establish working access into the pericardial space.

15. The method of claim 12 wherein the plurality of tines are disposed about a periphery of a distal end of the inner tube and wherein each tine is straightened when constrained in the outer tube and everts radially outwardly as it is advanced from the distal end of the outer tube.

16. A method for accessing a pericardial space between a visceral pericardium and a parietal pericardium surrounding a patient's heart, said method comprising:
percutaneously advancing a tissue-penetrating end of a cannula into the patient's thorax;
retracting the tissue-penetrating end of the cannula proximally over an inner tube to cause a plurality of tines to evert radially outwardly to form an atraumatic interface over a target site on an exterior of the parietal pericardium surrounding the patient's heart;
advancing the inner tube through the retracted cannula to engage the atraumatic interface against the target site on the exterior of the parietal pericardium surrounding the patient's heart;
advancing an access needle through an axial passage of the inner tube so that a distal tip of the access needle passes through the parietal pericardium to enter the pericardial space with minimal or no penetration visceral pericardium, wherein the needle has a lumen to provide an access path into the pericardial space.

17. The method of claim 16 wherein percutaneously advancing a tissue-penetrating end of a cannula into the patient's thorax comprises penetrating the cannula in a sub-xiphoid approach.

18. The method of claim 16 wherein percutaneously advancing a tissue-penetrating end of a cannula into the patient's thorax comprises penetrating the cannula in an intercostal approach.

19. The method of claim 16 further comprising advancing a guidewire through a lumen of the access needle to a target location in the peritoneal space.

20. The method of claim 19 further comprising withdrawing the cannula, inner tube, and access needle from over a proximal section of the guidewire and advancing an introducer sheath over the proximal section of the guidewire and through the parietal pericardium to establish working access into the pericardial space.

21. The method of claim 16 wherein the plurality of tines are disposed about a periphery of a distal end of the inner tube and wherein each tine is straightened when constrained in the outer tube and everts radially outwardly as it is advanced from the distal end of the outer tube.
